(19) United States
(12) Patent Application Publication
(10) Pub. No.: US 2016/0030373 A1
(43) Pub. Date: Feb. 4, 2016

(54) 2-AAA as a Biomarker and Therapeutic Agent for Diabetes

(71) Applicants: THE GENERAL HOSPITAL CORPORATION, Boston, MA (US); THE BROAD INSTITUTE, INC., Cambridge, MA (US)

(72) Inventors: Robert Gerszten, Brookline, MA (US); Thomas Wang, Lexington, MA (US); Clary Clish, Reading, MA (US)

(21) Appl. No.: 14/775,542

(22) PCT Filed: Mar. 10, 2014
(86) PCT No.: PCT/US14/22344

§ 371 Date: Sep. 11, 2015

Related U.S. Application Data
(60) Provisional application No. 61/780,172, filed on Mar. 13, 2013.

(51) Int. Cl. A61K 31/198; G01N 30/72; H01J 49/00; A61K 45/06; G01N 33/49

(52) U.S. Cl. CPC A61K 31/198; A61K 45/06; G01N 33/49; H01J 49/0027; G01N 30/7233; G01N 2800/52; G01N 2800/042

(57) ABSTRACT

Methods for treating a glucose-related metabolic disorder (e.g., diabetes) comprising administration of 2-aminoadipic acid (2-AAA) to subjects in need thereof. Also described are methods for predicting a subject's risk of developing a glucose-related metabolic disorder, and to methods for selecting and monitoring a treatment for a glucose-related metabolic disorder (e.g., diabetes).

### CLAIM OF PRIORITY

This application claims the benefit of, and incorporates by reference, U.S. Provisional Patent Applications Nos. 61/780,172, filed on Mar. 13, 2013.

### FEDERALLY SPONSORED RESEARCH OR DEVELOPMENT

This invention was made with Government support under Grant Nos. NO1-25195, and R01-DK-HL081572, awarded by the National Institutes of Health. The Government has certain rights in the invention.

### TECHNICAL FIELD

This invention relates to methods for treating glucose-related metabolic disorders using 2-aminoadipic acid (2-AAA), therapeutic compositions, and to methods for using metabolite biomarkers, e.g., 2-AAA, to determine the risk of diabetes, and to therapeutic compositions useful in the treatment of glucose-related metabolic disorders.

### BACKGROUND

The burden of type 2 diabetes mellitus is increasing, with an estimated 366 million cases worldwide. Type 2 diabetes mellitus develops slowly and, in some cases, the disease may not be detected before the onset of overt disease, where damage to eyes, kidneys or other organs has already occurred. Given the availability of proven interventions for delaying or preventing diabetes, early identification of individuals at risk is a public health priority (1-4). Therefore, there is an unmet need for metabolic biomarkers and tests that can identify subjects at risk for developing type 2 diabetes. Emerging technologies have enhanced the feasibility of acquiring detailed profiles of a human's metabolic status (metabolite profiling, or metabolomics) (5-9). Ongoing improvements in metabolomics technologies now provide sufficient sample throughput to make studies of epidemiological cohorts more feasible (6-9). These techniques, which allow the assessment of large numbers of metabolites that are substrates and products in metabolic pathways, have the potential to identify biochemical changes before the onset of overt clinical disease.

### SUMMARY

At least in part, the present invention is based on the discovery that 2-aminoadipic acid is a biomarker useful in identifying a subject's risk of developing a glucose-related metabolic disorder (e.g., diabetes), and that administration of therapeutic compositions comprising 2-aminoadipic acid is useful in the treatment of diabetes, e.g., for type 1 diabetes, type 2 diabetes, or gestational diabetes.

In one aspect, this disclosure provides methods for treating a glucose-related metabolic disorder in a subject in need thereof, the method comprising administering to the subject an effective amount of 2-aminoadipic acid (2-AAA). In one aspect, the glucose-related metabolic disorder is diabetes (e.g., type 1 diabetes, type 2 diabetes, or gestational diabetes). Administration of 2-AAA can be in the form of a pharmaceutical composition (e.g., tablets, granules, capsules, suspensions, solutions or injections). In one aspect, the method can further comprise administering one or more additional anti-diabetes compounds selected from the group consisting of acarbose, miglitol, metformin, phenformin, buformin, repaglinide, nateglinide, tolbutamide, chlorpropamide, tolazamide, acetohexamide, glyburide, glipizide, glimepiride, gliclazide, troglitazone, rosiglitazone, pioglitazone, peptide analogs, glucagon-like peptide I (GLP1) and analogs thereof (e.g., Exentide, Extendin-4, Liraglutide, gastric inhibitory peptide (GIP) and analogs thereof; vanadates (e.g., vanadyl sulfate), GLP agonists, vildagliptin sitagliptin; dichloroacetic acid; amylin, carnitine palmitoyltransferase inhibitors, B3 adrenoceptor agonists, and insulin.

In another aspect, this disclosure provides methods for method of increasing the level of pancreatic insulin secretion in a subject in need thereof, the method comprising administering to the subject an effective amount of 2-aminoadipic acid (2-AAA).

In a another aspect, this disclosure also provides a method of decreasing fasting glucose levels in a in a subject in need thereof, the method comprising administering to the subject an effective amount of 2-aminoadipic acid (2-AAA).

In a further aspect, the methods provided herein further comprise administering a treatment for a cardiovascular condition, e.g., a treatment selected from the group consisting of a hypolipidemic medication, a vasodilating compound, an anticoagulant, and sublingual glyceryl trinitrate, or any combination thereof.

In one aspect, this disclosure provides a method for determining risk of developing diabetes in a subject, the method comprising determining a level of 2-aminoadipic acid (2-AAA) in a test sample from the subject; comparing the level of 2-AAA in the test sample to a reference level; and determining the subject has an increased risk of developing diabetes when the test sample has an increased level of 2-AAA as compared to the reference level. The level of 2-aminoadipic acid can be determined using a mass spectrometer. In one aspect, an increase in the level of 2-aminoadipic acid in a test sample, as compared with the reference level, indicates an at least 3-fold, or an at least 4-fold increased risk of developing diabetes. The method can further comprise selecting a treatment based on the level of 2-aminoadipic acid in the test sample and administering said selected treatment to the subject. In one aspect, the treatment can comprise administering to the subject an effective amount of at least one anti-diabetes compound. In another aspect, the treatment can comprise administering to the subject an effective amount of 2-aminoadipic acid for increasing the level of insulin secretion.

In a further aspect, this disclosure provides a method for determining risk of developing diabetes in a subject, the method comprising determining a level of 2-aminoadipic acid (2-AAA) in a test sample from the subject; comparing the level of 2-AAA in the test sample to a reference level; and determining the subject has an increased risk of developing diabetes when the test sample has an increased level of 2-AAA as compared to the reference level; and further comprising determining the level of 2-AAA in a control sample from a control subject not having, or at risk of developing diabetes; comparing the level of 2-AAA in the test sample to the level of 2-AAA in the control sample; and determining the subject has an increased risk of developing diabetes when the test sample has an increased level of 2-AAA as compared to the level of 2-AAA in the control sample. In one aspect an increase in the level of 2-aminoadipic acid in a test sample, as compared with the control sample, indicates an at least 3-fold, or an at least 4-fold increased risk of developing diabetes.

Further provided herein is a kit for use in a method of determining risk of diabetes in a subject of claims 14-24, the kit comprising one or more control samples comprising predetermined levels of 2-aminoadipic acid.

In yet another aspect, the disclosure provides use of 2-aminoadipic acid in the manufacture of a medicament for the treatment of a glucose-related metabolic disorder (e.g., 2-aminoadipic acid for the treatment of a glucose-related metabolic disorder.)

The section headings used herein are for organizational purposes only and are not to be construed as limiting the described subject matter in any way. When definitions of terms in incorporated references appear to differ from the definitions provided in the present teachings, the definition provided in the present teachings shall control. It will be appreciated that there is an implied “about” prior to metrics such as temperatures, concentrations, and times discussed in the present teachings, such that slight and insubstantial deviations are within the scope of the present teachings herein. In this application, the use of the singular includes the plural unless specifically stated otherwise. Also, the use of “comprise,” “comprises,” “comprising,” “contain,” “contains,” “containing,” “include,” “includes,” and “including” are not intended to be limiting. It is to be understood that both the foregoing general description and the following detailed description are exemplary and explanatory only and are not restrictive of the invention. The articles “a” and “an” are used herein to refer to one or to more than one (i.e., to at least one) of the grammatical object of the article. By way of example, “an element” means one element or more than one element.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a graph depicting representative dose-response study using isotope-labeled standard for 2-aminoadipic acid (“2-AAA”) in normal pooled human plasma is shown. The parent to product ion MRM transition used for 2-AAA-d3 was m/z 163 to m/z 119, while the MRM transition for endogenous 2-AAA was 160 to 116. Boxes represent mean data from calibration curves run at the beginning, middle, and end of each analytical batch of ˜150 samples. The median concentration of the endogenous 2-AAA in the control samples as assessed by the LC-MS method is denoted with an arrow (1.22M). Peak areas were >2 orders of magnitude above the lower limit of quantitation (as defined as a discrete peak 10-fold greater than noise, lowest dose with a closed box) and fell well within the linear range of the dose-response relationship.

FIGS. 2A and B are graphs depicting fasting plasma glucose levels in 2-AAA in mice fed either a standard chow (2A) or high-fat diet (2B). Fasting plasma glucose levels were measured weekly in mice fed either a standard chow (left) or high-fat diet (right) beginning at 6 weeks of age, with simultaneous 2-AAA treatment via drinking water (500/mg/kg/day) or water alone for the subsequent 5 weeks. (n=24 mice per condition) (*p<0.05; **p<0.01; ***p<0.001).

FIGS. 3A and 3B are graphs depicting serial weights and food intake in control and 2-AAA treated animals.

FIG. 4 is a graph depicting plasma glucose levels in 2-AAA mice fed either a standard chow (left) or high-fat diet (right) following IPGTT challenge after completion of the 2-AAA chronic treatment in mice fed either the standard chow or high-fat diet. (n=12 mice per condition) (*p<0.05; **p<0.01).

FIG. 5 is a graph depicting fasting plasma insulin levels in 2-AAA mice fed either a standard chow (left) or high-fat diet (right) measured following completion of the 2-AAA treatment (5 weeks) in the mice on both diets (n=12 mice per condition) (*p<0.05).

FIGS. 6A and 6B are graphs depicting plasma glucose levels 2-AAA mice fed either a standard chow (left) or high-fat diet (right) measured following acute insulin challenge (n=15 per condition).

FIG. 7 is a graph depicting 2-AAA levels in the pancreas measured using an isotopically labeled standard. 2-AAA levels were increased following the administration of the high-fat diet, and further augmented following 2-AAA administration. (n=12 per condition) (***p<0.001).

FIG. 8A is a graph depicting percent insulin secretion by BTC6 cells following incubation with 2-AAA at concentrations ranging from 0 to 100 μM for 0.5 to 72 hours.

FIG. 8B is a graph depicting the effects of 2-AAA (30 μM), clonidine (100 μM) and phentolamine (100 μM) on insulin secretion by BTC6 cells. (*p<0.05, **p<0.01, ***p<0.001.)

FIG. 9 is a graph depicting insulin secretion from islets isolated from male C57BL/6J mice following incubation with 30 μM 2-AAA for 6 hours. Insulin was assayed using the Meso Scale Discovery multi array assay system for mouse/rat total insulin (Gaithersburg, Md., USA). Secretion was normalized to islet content. (n=3, p=0.016)

### DETAILED DESCRIPTION

The present inventors have developed a liquid chromatography-tandem mass spectrometry (LC/MS)-based platform capable of profiling 70 small molecules preferentially ionized using negative mode electrospray ionization, including intermediary organic acids, purines, pyrimidines and other compounds. This platform has been applied to the study of human plasma to identify metabolite biomarkers of diabetes risk, in two large, epidemiologic cohorts with more than a decade of follow up (e.g., the Framingham Offspring Study and the Malmö Diet and Cancer (MDC) study).

As described herein, the inventors applied this platform to complete a nested case-control study of 188 individuals who developed diabetes and 188 propensity-matched controls from 2,422 normoglycemic participants followed for 12 years in the Framingham Offspring Study. As demonstrated herein, the metabolite most strongly associated with the risk of developing diabetes was 2-aminoadipic acid (p=0.0009). Individuals with 2-AAA concentrations in the top quartile had >four-fold risk of developing diabetes (adjusted odds ratio, 4.5, 95% confidence interval, 1.9 to 10.9). These findings were replicated in the Malmö Diet and Cancer Study (p=0.004; pooled result, p<0.0001). Levels of 2-AAA were not well correlated with other metabolite biomarkers of diabetes, such as branched chain amino acids (r=0.04 to 0.24) and aromatic amino acids (r=0.01 to 0.13), suggesting they report on a distinct pathophysiological pathway. These data highlight a metabolite not previously associated with diabetes risk that is increased up to 12 years before the onset of overt disease. The experimental findings described herein also demonstrate higher 2-AAA levels in hyperinsulinemic mice fed a high-fat diet.

Furthermore, 2-AAA treatment enhanced insulin production by a pancreatic beta cell line, and administration of 2-AAA to mice leads to a significant decrease in fasting glucose levels in mice fed both standard chow and high fat diets. Metabolite profiling studies of tissues highlighted the pancreas as a potential organ of action for 2-AAA, and in vitro studies suggest that chronic administration of the metabolite increases beta cell insulin secretion. These data identify 2-AAA as a novel marker of diabetes risk and as a modulator of glucose homeostasis in humans.

### 2-Aminoadipic Acid

2-aminoadipic acid (“2-AAA” or “α-aminoadipic acid”) is a poorly characterized product of lysine degradation. The -amino group of lysine residues in proteins can undergo deamination by metal-catalyzed oxidation to form the intermediate allysine, which in turn undergoes further oxidation to form 2-aminoadipidic acid (10). 2-AAA may appear in the circulation from degradation of whole tissue or plasma proteins. Alternatively, 2-AAA might be generated from circulating lysine by some unknown enzymatic pathway.

2-AAA has the following structure:

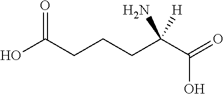 [SMILES: N[C@H](CCCC(=O)O)C(=O)O]

### Methods of Treatment

Disclosed herein are methods for treating a glucose-related metabolic disorder comprising administering a therapeutically effective amount of 2-AAA. The methods can include selection of a subject, e.g., selecting a subject for treatment according to a method described herein, e.g., by identifying a subject who has, or is at risk of developing, a glucose-related metabolic disorder as described herein.

As used in this context, to “treat” means to ameliorate at least one symptom or complication associated with the glucose-related metabolic disorder.

An “effective amount” is an amount sufficient to effect beneficial or desired results. For example, a therapeutic amount is one that treats the disorder or achieves a desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered from one or more times per day to one or more times per week; including once every other day. The skilled artisan will appreciate that certain factors may influence the dosage and timing required to effectively treat a subject, including, but not limited to, the severity of the disease or disorder, previous treatments, the general health and/or age of the subject, and other diseases present. Moreover, treatment of a subject with a therapeutically effective amount of the therapeutic compounds described herein can include a single treatment or a series of treatments.

The term “subject” as used herein refers to a mammal A subject therefore refers to, for example, dogs, cats, horses, cows, pigs, guinea pigs, and the like. The subject can be a human. When the subject is a human, the subject may be referred to herein as a patient.

Dosage, toxicity, and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are typically preferred. While compounds that exhibit toxic side effects may be used, care should be taken to design a delivery system that targets such compounds to the site of affected tissue to minimize potential damage to uninfected cells and, thereby, reduce side effects.

The data obtained from cell culture assays and animal studies can be used in formulating a range of dosages for use in humans. The dosage of such compounds lies preferably within a range of circulating concentrations that include the ED50 with little or no toxicity. The dosage may vary within this range depending upon the dosage form employed and the route of administration utilized. For any compound used in the methods of the inventions described herein, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Generally, the methods include administering a therapeutically effective amount of 2-aminoadipic acid to a subject who is in need of, or who has been determined to be in need of, such treatment.

The present methods can also be used for selecting a treatment and/or determining a treatment plan for a subject, based on the occurrence or levels of certain metabolite biomarkers (e.g., 2-AAA) relevant to the glucose-related metabolic disorders. In some embodiments, using the method disclosed herein, a health care provider (e.g., a physician) identifies a subject as having, or at risk of having or developing, a glucose-related metabolic disorder (e.g., Type II Diabetes) and, based on this identification the health care provider determines an adequate treatment plan for the subject. In some embodiments, using the method disclosed herein, a health care provider (e.g., a physician) diagnoses a subject as having, or at risk of having or developing, a glucose-related metabolic disorder (e.g., Type II Diabetes) based on the occurrence or levels of certain metabolite biomarkers (e.g., 2-AAA) in a clinical sample obtained from the subject, and/or based on a classification of a clinical sample obtained from the subject. By way of this diagnosis the health care provider determines an adequate treatment or treatment plan for the subject. In some embodiments, the methods further include administering the treatment to the subject.

In some embodiments, the invention relates to identifying subjects who are likely to have successful treatment with a particular drug dose, formulation and/or administration modality. In some embodiments, the metabolic profiling methods are useful for identifying subjects who are likely to have successful treatment with a particular drug or therapeutic regiment. For example, during a study (e.g., a clinical study) of a drug or treatment, subjects who have a glucose-related metabolic disorder may respond well to the drug or treatment, and others may not. Disparity in treatment efficacy is associated with numerous variables, for example genetic variations among the subjects. In some embodiments, subjects in a population are stratified based on the metabolic profiling methods disclosed herein. In some embodiments, resulting strata are further evaluated based on various epidemiological, and or clinical factors (e.g., response to a specific treatment). In some embodiments, stratum, identified based on a metabolic profile, reflect a subpopulation of subjects that response predictably (e.g., have a predetermined response) to certain treatments. In further embodiments, samples are obtained from subjects who have been subjected to the drug being tested and who have a predetermined response to the treatment. In some cases, a reference can be established from all or a portion of the metabolite biomarkers (e.g., 2-AAA) from these samples, for example, to provide a reference metabolic profile. A sample to be tested can then be evaluated (e.g., using a prediction model) against the reference and classified on the basis of whether treatment would be successful or unsuccessful. A company and/or person testing a treatment (e.g., compound, drug, life-style change) could discern more accurate information regarding the types or subtypes of glucose-related metabolic disorders for which a treatment is most useful. This information also aids a healthcare provider in determining the best treatment plan for a subject.

In some embodiments, treatment for the glucose-related metabolic disorder is to administer to the subject an effective amount of 2-aminoadipic acid and an effective amount of at least one additional anti-diabetes compound and/or to instruct the subject to adopt at least one anti-diabetic lifestyle change. Anti-diabetes compound are well known in the art and some are disclosed herein. Non-limiting examples include alpha-glucosidase inhibitors for example acarbose and miglitol; biguanides for example metformin, phenformin, and buformin; meglitinides for example, repaglinide and nateglinide; sulfonylureas, for example tolbutamide, chlorpropamide, tolazamide, acetohexamide, glyburide, glipizide, glimepiride, and gliclazide; thiazolidinediones, for example troglitazone, rosiglitazone, and pioglitazone; peptide analogs, for example glucagon-like peptide I (GLP1) and analogs thereof (e.g., Exentide, Extendin-4, Liraglutide, gastric inhibitory peptide (GIP) and analogs thereof; vanadates (e.g., vanadyl sulfate); GLP agonists; DPP-4 inhibitors, for example vildagliptin and sitagliptin; dichloroacetic acid; amylin; carnitine palmitoyltransferase inhibitors; B3 adrenoceptor agonists; and insulin. Appropriate anti-diabetic lifestyle changes are also well known in the art. Non-limiting examples include increased physical activity, caloric intake restriction, nutritional meal planning, and weight reduction. However, the invention is not so limited and other appropriate treatments will be apparent to one of ordinary skill in the art.

When a therapeutic agent (e.g., anti-diabetic compound) or other treatment is administered, it is administered in an amount effective to treat an existing glucose-related metabolic disorder or reduce the likelihood (or risk) of a future glucose-related metabolic disorder. An effective amount is a dosage of the therapeutic agent sufficient to provide a medically desirable result. The effective amount will vary with the particular condition being treated, the age and physical condition of the subject being treated, the severity of the condition, the duration of the treatment, the nature of the concurrent therapy (if any), the specific route of administration and the like factors within the knowledge and expertise of the health care practitioner. For example, an effective amount can depend upon the degree to which a subject has abnormal levels of certain metabolite biomarkers (e.g., 2-AAA) that are indicative of presence or risk a glucose-related metabolic disorder. It should be understood that the therapeutic agents of the invention are used to treat and/or prevent glucose-related metabolic disorders. Thus, in some cases, they may be used prophylactically in human subjects at risk of developing a glucose-related metabolic disorder. Thus, in some cases, an effective amount is that amount which can lower the risk of, slow or perhaps prevent altogether the development of a glucose-related metabolic disorder. It will be recognized when the therapeutic agent is used in acute circumstances, it is used to prevent one or more medically undesirable results that typically flow from such adverse events.

In some embodiments, the invention relates to methods for the treatment of subjects with diabetes, e.g., Type 1 or Type 2 diabetic subjects with cardiovascular disease (e.g., atherosclerosis, hypercholesterolemia) or myocardial infarction. The treatment can comprise administration of 2-AAA and at least one additional anti-diabetic agent, hypolipidemic medication, vasodilating compound, anticoagulant, and sublingual glyceryl trinitrate, or any combination thereof. Thus, further to the treatment for the glucose-related metabolic disorder, the treatment can be to cure, heal, alleviate, relieve, alter, remedy, ameliorate, palliate, improve or affect cardiovascular disease or myocardial infarction. For example, a standard treatment regimen for myocardial infarction can include administering anticoagulant or vasodilating compounds, administering sublingual glyceryl trinitrate (nitroglycerin), and/or administering pain relief. Preventative therapeutic measures can additionally or alternatively include administering hypolipidemic medications (e.g., statins, including, for example, atorvastatin, simvastatin, pravastatin, rivastatin, mevastatin, fluindostatin, velostatin, fluvastatin, dalvastatin, dihydrocompactin, compactin, cerivastatin or lovastatin), promoting diet and exercise, and promoting weight loss. A standard treatment regimen for atherosclerosis can include administering anticoagulant or vasodilating compounds, administering hypolipidemic medications, performing balloon angioplasty, or performing artery bypass surgery. Standard therapeutic strategies for hypercholesterolemia include administering hypolipidemic medications, promoting diet and exercise, and promoting weight loss.

After one or more doses of a treatment have been administered, the present methods can be used to monitor efficacy, wherein an increase in a level or ratio of 2-AAA is associated with increased risk, or a decrease in a level or ratio of a 2-AAA is associated with decreased risk, would indicate that the treatment is effective in reducing risk. Methods for selecting a suitable treatment and an appropriate dose thereof will be apparent to one of ordinary skill in the art.

Glucose-Related Metabolic Disorders

The invention, in some aspects, relates to methods, compositions and kits useful for treatment, diagnosing and determining risk of developing glucose-related metabolic disorders. As used herein, “glucose-related metabolic disorders” refer broadly to any disorder, disease, or syndrome characterized by a deficiency in the regulation of glucose homeostasis (e.g., hyperglycemia). Typically a glucose-related metabolic disorder is associated with abnormal insulin levels, insulin activity, and/or sensitivity to insulin (e.g., insulin resistance). As used herein diabetes (also referred to as diabetes mellitus), refers to any one of a number of exemplary classes (or types) of glucose-related metabolic disorders. Diabetes includes, but is not limited to the following classes (or types): type I diabetes mellitus, type II diabetes mellitus, gestational diabetes, and other specific types of diabetes. Glucose-related metabolic disorders also include prediabetic conditions, such as those associated with impaired fasting glycemia and impaired glucose tolerance. Glucose-related metabolic disorders are often associated with symptoms in a subject such as increased thirst and urine volume, recurrent infections, unexplained weight loss and, in severe cases, drowsiness and coma; high levels of glycosuria are often present. Children suspected of having a glucose-related metabolic disorder may, in some cases, present with severe symptoms, such as high blood glucose levels, glycosuria, and/or ketonuria.

Type 1 diabetes is usually due to autoimmune destruction of the pancreatic beta cells. Type 2 diabetes is characterized by insulin resistance in target tissues, which may result in a need for abnormally high amounts of insulin and diabetes develops when the beta cells cannot meet this demand Gestational diabetes is similar to type 2 diabetes in that it involves insulin resistance; the hormones of pregnancy can cause insulin resistance in women genetically predisposed to developing this condition. Other specific types of diabetes are known in the art and disclosed in Definition, Diagnosis and Classification of Diabetes Mellitus and its Complications, Report: WHO/NCD/NCS/99.2 by the World Health Organization, Department of Noncommunicable Disease Surveillance (1999), the contents of which are incorporated herein in their entirety by reference.

In some embodiments, the glucose-related metabolic disorder is Type 2 diabetes. Type 2 is also referred to as non-insulin-dependent diabetes or adult-onset diabetes, and is characterized by disorders of insulin action and insulin secretion, either of which may be the predominant feature. Both are usually present at the time that this form of diabetes is clinically manifest.

In some embodiments, the glucose-related metabolic disorder is gestational hyperglycemia or gestational diabetes. These are forms of diabetes associated with pregnancy. Gestational diabetes is associated with carbohydrate intolerance resulting in hyperglycemia of variable severity with onset or first recognition during pregnancy. Thus, it does not exclude the possibility that the glucose intolerance may antedate the pregnancy but was previously unrecognized. The classification typically applies irrespective of whether or not insulin is used for treatment or the condition persists after pregnancy.

In some embodiments, the glucose-related metabolic disorder is “Metabolic Syndrome” which is often characterized by hypertension, central (upper body) obesity, and dyslipidaemia, with or without hyperglycaemia. Subjects with the Metabolic Syndrome are at high risk of macrovascular disease. Often a person with abnormal glucose tolerance will be found to have at least one or more of the other cardiovascular disease (CVD) risk components. The Metabolic Syndrome is also referred to as Syndrome X and the Insulin Resistance Syndrome. Epidemiological studies confirm that this syndrome occurs commonly in a wide variety of ethnic groups including Caucasians, African-Americans, Mexican-Americans, Asian Indians, Chinese, Australian Aborigines, Polynesians and Micronesians. The Metabolic Syndrome with normal glucose tolerance identifies a subject as a member of a group at very high risk of diabetes. Thus, vigorous early management of the syndrome may have a significant impact on the prevention of both diabetes and cardiovascular disease.

Diagnosis/Characterization

The present invention relates to methods useful for the characterization (e.g., clinical evaluation, diagnosis, classification, prediction, profiling) of glucose-related metabolic disorders, such as diabetes, based on the levels, presence, or absence of certain metabolite biomarkers (e.g., 2-AAA). As used herein, levels refer to the amount or concentration of a metabolite biomarkers in a sample (e.g., a plasma sample) or subject. The level may be expressed as an exact quantity, or may be expressed as a ratio to a reference 2-AAA sample. In some cases, the methods can include determining whether 2-AAA is present in a concentration or a ratio above or below a reference level or ratio.

In some embodiments, the methods involve determining the ratio or levels of one or a plurality of metabolite biomarkers (e.g., 2-AAA) in a clinical sample, comparing the result to a reference ratio or level, and characterizing (e.g., diagnosing, classifying) the sample based on the results of the comparison. A clinical sample can be any biological specimen (e.g., a blood sample) useful for characterizing the glucose-related metabolic disorder (e.g., diabetes). Exemplary biological specimens can include blood, serum, or plasma. In preferred embodiments, a clinical sample is a plasma sample.

In some embodiments, clinical samples are obtained from subjects (also referred to herein as individuals). As used herein, a subject is a mammal, including but not limited to a dog, cat, horse, cow, pig, sheep, goat, chicken, rodent, or primate (e.g., a human). Subjects can be house pets (e.g., dogs, cats), agricultural stock animals (e.g., cows, horses, pigs, chickens, etc.), laboratory animals (e.g., mice, rats, rabbits, etc.), zoo animals (e.g., lions, giraffes, etc.), but are not so limited. In some embodiments, a subject is a diabetic animal model. Diabetes animal models are well known in the art, for example: Leiter, Curr Protoc Immunol. 2001 May; Chapter 15:Unit 15.9; Levine et al., Am J Physiol Regul Integr Comp Physiol. 2008 Apr. 16; Oh Y S, et al., Diabetologia. 2008 Apr. 12; Sasaki et al., Arterioscler Thromb Vasc Biol. 2008 Apr. 10; Beauquis et al., Exp Neurol. 2008 April; 210(2):359-67; Cheng et al., Mol Pharm. 2008 January-February; 5(1):77-91; Tikellis et al., Atherosclerosis. 2007 Dec. 17; Novelli et al., Pancreas. 2007 November; 35(4):e10-7; and Khazaei et al., Physiol Res. 2007 Nov. 30. Preferred subjects are humans (human subjects). The human subject may be a pediatric or adult subject. In some embodiments the adult subject is an overweight (BMI of 25-29) or obese (BMI of 30 or higher) subject.

In some embodiments, the methods involve diagnosing glucose-related metabolic disorder in a subject. To practice the diagnostic methods the levels of a plurality of biomarkers are typically determined. These levels are compared to a reference wherein the levels of the plurality of biomarkers in comparison to the reference is indicative of whether or not the subject has a glucose-related metabolic disorder and/or should be diagnosed with the glucose-related metabolic disorder.

As used herein, diagnosing includes both diagnosing and aiding in diagnosing. Thus, other diagnostic criteria may be evaluated in conjunction with the results of the methods herein in order to make a diagnosis.

The methods described herein are also useful for assessing the likelihood (or risk) of, or aiding in assessing the likelihood (or risk) of, a subject having or developing a glucose-related metabolic disorder. To practice the methods levels of a plurality of biomarkers are typically determined. These levels are compared to a reference wherein the levels or ratios of the plurality of biomarkers in comparison to the reference levels or ratios is indicative of the likelihood that the subject will develop a glucose-related metabolic disorder.

Other criteria for assessing likelihood that are known in the art (e.g., Body Mass Index (BMI), family history) can also be evaluated in conjunction with the methods described herein in order to make a complete likelihood assessment.

In some embodiments, methods involve determining the glucose control capacity or insulin sensitivity of a subject. To practice the methods, typically the levels of a plurality of biomarkers are determined. These levels are compared to a reference wherein the levels of the plurality of biomarkers in comparison to the reference are indicative of the glucose control capacity or insulin sensitivity.

As used herein, insulin sensitivity refers to the responsiveness of a subject, or cells of a subject, to the effects of insulin. For example, subjects with insulin resistance are less sensitive to insulin and therefore, have low insulin sensitivity. Techniques for measuring insulin sensitivity are well known in the art and include, for example, the hyperinsulinemic euglycemic clamp (i.e., the “clamp” technique), the Modified Insulin Suppression Test, fasting insulin levels, and glucose tolerance tests (e.g., an Oral Glucose Tolerance Test). The methods disclosed herein are also useful to characterize and obtain further insight on insulin sensitivity.

As used herein, glucose control capacity refers to a subject's ability (capacity) to control glucose levels within homeostatic limits (a physiologically safe/normal range). Consequently, insulin (and therefore insulin sensitivity), among other things, influences a subject's glucose control capacity. Other regulatory factors (e.g., hormones) in addition to insulin, such as glucagon, that influence glucose control capacity of a subject are well known in art. The methods disclosed herein are useful to characterize and obtain further insight on glucose control capacity.

The level of the 2-AAA for a subject can be obtained by any art recognized method. Typically, the level is determined by measuring the level of 2-AAA in a sample comprising plasma, or serum. The level can be determined by any method known in the art, e.g., enzymatic assays, spectrophotometry, colorimetry, fluorometry, bacterial assays, liquid chromatography, gas chromatography, mass spectrometry, gas chromatography-mass spectrometry (GC-MS), liquid chromatography-mass spectrometry (LC-MS), LC-MS/MS, tandem MS, high pressure liquid chromatography (HPLC), HPLC-MS, and nuclear magnetic resonance spectroscopy, or other known techniques for determining the presence and/or quantity of 2-AAA; in some embodiments, the level is determined using one of LC-MS, HPLC-MS, or GC-MS. See, e.g., Suhre et al., Metabolic Footprint of Diabetes: A Multiplatform Metabolomics Study in an Epidemiological Setting. PLoS ONE 5(11): e13953 (2010). Conventional methods include sending a clinical sample(s) to a clinical laboratory, e.g., on site or a third party contractor, e.g., a commercial laboratory, for measurement.

In some cases, the methods disclosed herein involve comparing levels or occurrences (e.g., presence or absence) to a reference. The reference can take on a variety of forms. In some cases, the reference comprises predetermined values for a metabolite biomarker in a sample (e.g., 2-AAA). The predetermined value can take a variety of forms. It can be a level or occurrence of a 2-aminoadipic acid in a control subject (e.g., a subject with a glucose-related metabolic disorder (i.e., an affected subject) or a subject without such a disorder (i.e., a normal subject)). It can be a level or occurrence of a 2-aminoadipic acid in a fasting subject. It can be a level or occurrence in the same subject, e.g., at a different time point. A predetermined value that represents a level(s) of a 2-aminoadipic acid is referred to herein as a predetermined level. A predetermined level can be single cut-off value, such as a median or mean. It can be a range of cut-off (or threshold) values, such as a confidence interval. It can be established based upon comparative groups, such as where the risk in one defined group is a fold higher, or lower, (e.g., approximately 2-fold, 4-fold, 8-fold, 16-fold or more) than the risk in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

Subjects associated with predetermined values are typically referred to as control subjects (or controls). A control subject may or may not have a glucose-related metabolic disorder (e.g., diabetes). In some cases it may be desirable that control subject is a diabetic, and in other cases it may be desirable that a control subject is a non-diabetic. Thus, in some cases the level of the metabolite biomarker (e.g., 2-AAA) in a subject being greater than or equal to the level of the metabolite biomarker (e.g., 2-AAA) in a control subject is indicative of a clinical status (e.g., indicative of a glucose-related metabolic disorder diagnosis). In other cases the level of the metabolite biomarker (e.g., 2-AAA) in a subject being less than or equal to the level of the metabolite biomarker (e.g., 2-AAA) in a control subject is indicative of a clinical status. The amount of the greater than and the amount of the less than is usually of a sufficient magnitude to, for example, facilitate distinguishing a subject from a control subject using the disclosed methods. Typically, the greater than, or the less than, that is sufficient to distinguish a subject from a control subject is a statistically significant greater than, or a statistically significant less than. In cases where the level of the metabolite biomarker (e.g., 2-AAA) in a subject being equal to the level of the metabolite biomarker (e.g., 2-AAA) in a control subject is indicative of a clinical status, the “being equal” refers to being approximately equal (e.g., not statistically different).

The predetermined value can depend upon a particular population of subjects (e.g., human subjects) selected. For example, an apparently healthy population will have a different ‘normal’ range of the metabolite biomarker (e.g., 2-AAA) than will a population of subjects which have, or are likely to have, a glucose-related metabolic disorder. Accordingly, the predetermined values selected may take into account the category (e.g., healthy, at risk, diseased) in which a subject (e.g., human subject) falls. Appropriate ranges and categories can be selected with no more than routine experimentation by those of ordinary skill in the art.

In some cases a predetermined value of a metabolite biomarker is a value that is the average for a population of healthy subjects (human subjects) (e.g., human subjects who have no apparent signs and symptoms of a glucose-related metabolic disorder). The predetermined value will depend, of course, on the particular metabolite (biomarker) selected and even upon the characteristics of the population in which the subject lies. In characterizing likelihood, or risk, numerous predetermined values can be established.

A level, in some embodiments, may itself be a relative level that reflects a comparison of levels between two states. For example, a level may be a relative level that reflects a comparison between fasting (e.g., pre-glucose consumption) and non-fasting states (e.g., post-glucose consumption). Where levels are relative levels that reflect a comparison between fasting and non-fasting states, the non-fasting state may be, for example, about 30 minutes, about 60 minutes, about 90 minutes, about 120 minutes, or more, post glucose consumption. In some cases, relative levels may be determined (e.g., by clinical personnel) during a standard oral glucose tolerance test, e.g., a first or baseline level that is obtained before the test and a second level that is obtained after the glucose consumption). Relative levels that reflect a comparison (e.g., ratio, difference, logarithmic difference, percentage change, etc.) between two states (e.g., fasting and non-fasting) may be referred to as delta values. For example, in the case of an oral glucose tolerance test, delta values may be a percentage change in levels of a biomarker from fasting to non-fasting states. The use of relative levels is beneficial in some cases because, to an extent, they exclude measurement related variations (e.g., laboratory personnel, laboratories, measurements devices, reagent lots/preparations, assay kits, etc.). However, the invention is not so limited.

Pharmaceutical Compositions and Methods of Administration

The methods described herein include the manufacture and use of pharmaceutical compositions that include 2-aminoadipic acid, for use in a method of treatment as described herein.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions, e.g., anti-diabetes compounds, such as, for example alpha-glucosidase inhibitors for example acarbose and miglitol; biguanides for example metformin, phenformin, and buformin; meglitinides for example, repaglinide and nateglinide; sulfonylureas, for example tolbutamide, chlorpropamide, tolazamide, acetohexamide, glyburide, glipizide, glimepiride, and gliclazide; thiazolidinediones, for example troglitazone, rosiglitazone, and pioglitazone; peptide analogs, for example glucagon-like peptide I (GLP1) and analogs thereof (e.g., Exentide, Extendin-4, Liraglutide, gastric inhibitory peptide (GIP) and analogs thereof; vanadates (e.g., vanadyl sulfate); GLP agonists; DPP-4 inhibitors, for example vildagliptin and sitagliptin; dichloroacetic acid; amylin; carnitine palmitoyltransferase inhibitors; B3 adrenoceptor agonists; and insulin.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

Kits

The invention also provides kits for evaluating biomarkers in a subject. The kits of the invention can take on a variety of forms. Typically, the kits will include reagents suitable for determining levels of a plurality of biomarkers (e.g., those disclosed herein, for example as outlined in Table 1) in a sample. Optionally, the kits may contain one or more control samples or references. Typically, a comparison between the levels of the biomarkers in the subject and levels of the biomarkers in the control samples is indicative of a clinical status (e.g., diagnosis, likelihood assessment, insulin sensitivity, glucose control capacity, etc.). Also, the kits, in some cases, will include written information (indicia) providing a reference (e.g., predetermined values), wherein a comparison between the levels of the biomarkers in the subject and the reference (pre-determined values) is indicative of a clinical status. In some cases, the kits comprise software useful for comparing biomarker levels or occurrences with a reference (e.g., a prediction model). Usually the software will be provided in a computer readable format such as a compact disc, but it also may be available for downloading via the internet. However, the kits are not so limited and other variations with will apparent to one of ordinary skill in the art.

### Examples

The invention is further described in the following examples, which do not limit the scope of the invention described in the claims.

Statistical analyses for the following examples were performed as follows. For human studies, metabolite concentrations were log transformed to reduce heteroscedasticity of case-control differences. Initially, cases were compared with propensity-matched controls using paired t-tests. We considered metabolite findings with a p-value less than 0.01 to take to replication analyses. For the studies conditional (matched-pairs) logistic regression analyses were performed relating baseline metabolite values with future diabetes risk. Metabolites were treated as continuous and as categorical variables. We adjusted for age, sex, BMI, and fasting glucose. In additional analyses, we further adjusted for parental history, serum triglycerides, HDL cholesterol, hypertension, intake of dietary protein, amino acids, and total calories. A Bonferroni-corrected p-value threshold 7×10−4 (=0.05/70) was used to denote significance in the pooled analyses.

Pearson correlations were calculated between metabolite concentrations and other biochemical measures of insulin action: fasting insulin, homeostasis model assessment of insulin resistance (HOMA-IR) and -cell function (HOMA-B) (25). We then assessed whether metabolite concentrations predicted risk incrementally over these other biochemical measures. All analyses in the human cohorts were performed using SAS Statistical Software (version 9.3, Cary, N.C.).

For the animal studies, all data are expressed as means with error bars showing standard errors of the mean. Comparison of endpoints was performed using an unpaired two-tailed Student's t test. For the time course studies one-way ANOVA with repeated measurements was used. A p value of <0.05 was considered significant.

For the cell culture studies, the 2-AAA dose response was evaluated by an unpaired one-way ANOVA using Dunnet's multiple comparison test to determine level of significance of individual 2-AAA doses. An unpaired t-test using Welch correction for unequal variances was used to compare differences between control and clonidine, phentolamine, and 2-AAA, respectively. A p value of <0.05 was considered significant. All analyses for the animal and cell culture studies were performed using GraphPad Prism (v. 5.02, La Jolla Calif.).

### Example 1

### 2-Aminoadipic Acid (2-AAA) Predicts Future Diabetes

Plasma samples were obtained from two cohorts. The discovery analyses were performed on individuals from the Framingham Offspring Study (FHS), which was initiated in 1971 when 5,124 individuals enrolled into this longitudinal cohort study (22). Samples came from the 5th examination between 1991 and 1995. Of the 3,799 attendees of the 5^{th} examination (referred to as the baseline examination), metabolite profiling was performed on samples from 1,937 attendees who were free of diabetes (i.e., fasting glucose<126 mg/dl and not on glucose-lowering medications) at baseline (376 propensity-matched cases and controls, and 1,561 randomly-selected individuals). At each subsequent quadrennial visit, participants underwent a physician-administered physical examination and medical history, and routine laboratory tests.

The studies described herein utilized a methodology similar to the previously reported technique for profiling polar plasma metabolites using hydrophilic interaction liquid chromatography (HILIC) and tandem mass spectrometry (LC-MS)(8), though for this analysis the instant studies focused on small molecules preferentially ionized using negative mode electrospray ionization under basic conditions. Data were acquired using an ACQUITY UPLC (Waters, Milford Mass.) coupled to a 5500 QTRAP triple quadrupole mass spectrometer (AB SCIEX, Framingham, Mass.). To develop the method, chromatographic retention times and multiple reaction monitoring (MRM) MS settings were determined for more than 150 reference compounds, of which 70 could be detected in human plasma in the archived Framingham samples. 41 of the 70 metabolites were detectable in >99% of the human samples. Samples were prepared by the addition of 120 μL of extraction solution (80% methanol (VWR) plus the internal standards inosine-15N4, thymine-d4, and glycocholate-d4 (Cambridge Isotope Laboratories, Andover Mass.) to 30 microliters of plasma. The samples were centrifuged (10 min, 9,000×g, 4° C.) and the supernatants were injected directly onto a 150×2.0 mm Luna NH2 column (Phenomenex) that was eluted at a flow rate of 400 μL/min with initial conditions of 10% mobile phase A ((20 mM ammonium acetate and 20 mM ammonium hydroxide (Sigma-Aldrich, St. Louis Mo.) in water (VWR)) and 90% mobile phase B ((10 mM ammonium hydroxide in 75:25 v/v acetonitrile/methanol (VWR)) followed by a 10 min linear gradient to 100% mobile phase A. The ion spray voltage was −4.5 kV and the source temperature was 500° C. Raw data were processed using MultiQuant 1.2 (AB SCIEX). Data were normalized relative to pooled plasma reference samples that were analyzed in the sample queue after sets of 20 study samples.

The human study protocols for metabolite profiling were approved by the Institutional Review Boards of Boston University Medical Center, Massachusetts General Hospital, and Lund University, Sweden, and all participants provided written informed consent.

Baseline clinical characteristics are shown in Table 1. Cases and controls were similar with respect to age, sex, body mass index (BMI), and fasting glucose.

TABLE 1
Baseline characteristics

| | Framingham Heart Study | | | Malmö Diet and | | |
|---|---|---|---|---|---|---|
| | | | Additional | Cancer Study | | |
| | | Matched | Random | Whole | | Matched |
| | Cases | Controls | Cohort | Cohort | Cases | Controls |
| | (n = 188) | (n = 188) | (n = 1,561) | (n = 1,937) | (n = 162) | (n = 162) |
| Clinical characteristics | | | | | | |
| Age, years | 56 ± 9 | 57 ± 8 | 55 ± 10 | 55 ± 10 | 58 ± 6 | 58 ± 6 |
| Women, % | 43% | 43% | 54% | 52% | 55% | 55% |
| Body mass index, kg/m² | 30.5 ± 5.0 | 30.0 ± 5.5 | 26.7 ± 4.4 | 27.4 ± 4.8 | 28.2 ± 4.8 | 28.5 ± 4.9 |
| Waist circumference, | 102 ± 12 | 100 ± 14 | 91 ± 14 | 93 ± 14 | 91 ± 14 | 91 ± 16 |
| cm | | | | | | |
| Hypertension, % | 53% | 53% | 30% | 34% | 77% | 74% |
| Parental history of | 32% | 18% | 19% | 20% | 7% | 2% |
| diabetes*, % | | | | | | |
| Physical activity index | 36 ± 6 | 35 ± 7 | 35 ± 6 | 35 ± 6 | — | — |
| Total caloric intake, | 1,988 ± 658 | 1,862 ± 601 | 1,854 ± 611 | 1,868 ± 616 | — | — |
| kcal | | | | | | |
| Total protein intake, g | 82 ± 27 | 77 ± 28 | 77 ± 27 | 77 ± 27 | — | — |
| Lysine intake, g | 6 ± 2 | 6 ± 2 | 5 ± 2 | 6 ± 2 | — | — |
| Fasting glucose, mg/dl | 105 ± 9 | 105 ± 9 | 93 ± 9 | 96 ± 10 | 97 ± 8 | 97 ± 7 |

Values are mean ± SD, or percentage.
*Parental history information missing in 57 participants in Framingham sample.

From a screen of 70 metabolites, 2-AAA had the strongest association with future diabetes (p=0.0009, with a higher fasting concentration in the cases). Results for all metabolites profiled are shown in Table 2. The 57 metabolites listed in Table 2 were detected in at least 70% of the study samples.

TABLE 2
Metabolite profiling in individuals with and without
incident diabetes (Framingham Heart Study).

| | Paired | |
|---|---|---|
| Metabolite | T-statistic | P-value |
| 2-aminoadipate | 3.39 | 0.0009 |
| quinolinate | 2.53 | 0.0121 |
| PEP | 2.49 | 0.0138 |
| UDP-galactose/UDP-glucose | 2.42 | 0.0164 |
| hippurate | −2.19 | 0.0294 |
| F1P/F6P/G1P/G6P | 2.24 | 0.0265 |
| beta-hydroxybutyrate | −1.95 | 0.0529 |
| UDP | 1.91 | 0.0583 |
| 3-methyladipate | −1.85 | 0.0657 |
| salicylurate | 1.77 | 0.0780 |
| isocitrate | 1.61 | 0.11 |
| alpha-glycerophosphate | 1.58 | 0.12 |
| kynurenine | 1.56 | 0.12 |
| hypoxanthine | −1.44 | 0.15 |
| urate | 1.43 | 0.15 |
| glycodeoxycholate/glycochenodeoxycholate | 1.36 | 0.18 |
| glycocholate | 1.31 | 0.19 |
| 4-pyridoxate | −1.26 | 0.21 |
| phosphoglycerate | 1.23 | 0.22 |
| lactate | 1.13 | 0.26 |
| hydroxyphenylacetate | 1.13 | 0.26 |
| pantothenate | −1.09 | 0.28 |
| adipate | −0.99 | 0.32 |
| xanthurenate | 0.96 | 0.34 |
| fumarate/maleate | −0.91 | 0.36 |
| indole-3-propionate | −0.90 | 0.37 |
| alpha-ketoglutarate | −0.88 | 0.38 |
| xanthine | 0.78 | 0.44 |
| citrate | −0.76 | 0.45 |
| GDP | 0.75 | 0.45 |
| alpha-hydroxybutyrate | −0.74 | 0.46 |
| GMP | 0.73 | 0.46 |
| indoxylsulfate | 0.71 | 0.48 |
| uridine | 0.65 | 0.52 |
| cystathionine | 0.64 | 0.53 |
| ribose-5-phosphate/ribulose-5-phosphate | 0.63 | 0.53 |
| pyruvate | 0.56 | 0.57 |
| sucrose | 0.54 | 0.59 |
| Oxalate | −0.43 | 0.67 |
| hyodeoxycholate/ursodeoxycholate/ | 0.41 | 0.68 |
| chenodeoxycholate/deoxycholate | | |
| suberate | −0.34 | 0.74 |
| gentisate | 0.30 | 0.76 |
| aconitate | 0.29 | 0.77 |
| inositol | −0.29 | 0.77 |
| inosine | 0.26 | 0.79 |
| taurocholate | −0.26 | 0.80 |
| ADP | 0.26 | 0.80 |
| propionate | 0.25 | 0.80 |
| AMP | 0.25 | 0.81 |
| orotate | 0.18 | 0.86 |
| phosphocreatine | 0.15 | 0.88 |
| lactose | 0.13 | 0.90 |
| cAMP | −0.13 | 0.92 |
| taurodeoxycholate/taurochenodeoxycholate | 0.09 | 0.93 |
| 2-hydroxyglutarate | −0.09 | 0.93 |
| malate | −0.08 | 0.94 |
| sorbitol | 0.04 | 0.97 |

Results are from paired t-tests (case minus control) for each variable.

For replication, discovery analyses were also performed on individuals from the Malmö Diet and Cancer (MDC) study, a Swedish population-based cohort of 28,449 persons enrolled between 1991 and 1996. From this group, 6,103 persons were randomly selected to participate in the MDC Cardiovascular Cohort (23). We obtained fasting plasma samples in 5,305 subjects in the MDC Cardiovascular Cohort, of whom 564 had prevalent diabetes or cardiovascular disease prior to baseline. Of note, 456 subjects had missing covariate data, leaving 4,285 subjects eligible for analysis. Detailed descriptions of the clinical assessment, diabetes definition, and subject selection have been previously described (8).

For the MDC replication study, and as in the FHS, concentrations of 2-AAA were significantly higher in cases compared with matched controls (p=0.004; pooled p<0.0001). There was a 57% increased odds of future diabetes per SD increment in 2-AAA (p=0.004), nearly identical to that found in FHS (Table 2). Individuals in the top quartile had an adjusted odds for incident diabetes of 3.96 (95% CI, 1.63 to 9.59).

Conditional logistic regression models were performed adjusting for age, sex, BMI, and fasting glucose (Table 3). Each SD increment in log marker was associated with a 60% increased odds of future diabetes (p=0.002). Individuals in the top quartile of plasma 2-AAA concentration had a four-fold higher odds of developing diabetes over the 12-year follow-up period, compared with those in the lowest quartile (adjusted odds ratio 4.49, 95% CI, 1.86 to 10.89). Results were similar after further adjustment for parental history of diabetes, total caloric intake, and dietary protein, fat, or carbohydrates (data not shown). There was no interaction between follow-up year and the case-control difference for 2-AAA (p>0.10), suggesting a stable association with new-onset diabetes during the follow-up period. The association with 2-AAA was similar in analyses restricted to diabetes cases diagnosed 8 or more years after the baseline examination. The odds ratio for individuals in the highest quartile of 2-AAA was 4.16 (95% CI, 1.26-13.8).

TABLE 3
2-AAA and the risk of future diabetes
2-AAA

| | FHS | MDC | |
|---|---|---|---|
| | (188 cases, | (162 cases, | Combined |
| | 188 controls) | 162 controls) | sample |
| | 12-year | 13-year | (350 cases, |
| Model | follow-up | follow-up | 350 controls) |
| As | | | |
| continuous | | | |
| variable | | | |
| Per SD | 1.60 (1.19-2.16) | 1.57 (1.15-2.14) | 1.59 (1.28-1.97) |
| increment | | | |
| P | 0.002 | 0.004 | <0.0001 |
| As | | | |
| categorical | | | |
| variable | | | |
| 1st quartile | 1.00 (Referent) | 1.00 (Referent) | 1.00 (Referent) |
| 2nd quartile | 1.34 (0.72-2.49) | 2.19 (1.07-4.48) | 1.66 (1.05-2.63) |
| 3rd quartile | 1.71 (0.82-3.54) | 1.45 (0.68-3.07) | 1.56 (0.93-2.61) |
| 4th quartile | 4.49 (1.86-10.89) | 3.96 (1.63-9.59) | 4.12 (2.22-7.65) |
| P for trend | 0.001 | 0.01 | <0.0001 |

Values are odds ratios (95% confidence intervals) for diabetes, from conditional logistic regressions. All models are adjusted for age, sex, BMI, and fasting glucose. For the test of linear trend, quartiles were assigned values of 1, 2, 3, and 4.

Tables 2 and 3 each show that the 2-AAA is a novel metabolite biomarker that predicts the development of diabetes in normoglycemic individuals. Individuals with 2-AAA concentrations in the top quartile had >four-fold risk of developing diabetes (adjusted odds ratio, 4.5, 95% confidence interval, 1.9 to 10.9). These findings were replicated in the Malmö Diet and Cancer Study (p=0.004; pooled result, p<0.0001). Levels of 2-AAA were not well correlated with other metabolite biomarkers of diabetes, such as branched chain amino acids (r=0.04 to 0.24) and aromatic amino acids (r=0.01 to 0.13), suggesting they report on a distinct pathophysiological pathway.

The case-control analyses were enriched for individuals with “high risk” features, such as obesity and elevated fasting glucose. Thus, to assess the generalizability of the results in a more heterogeneous cohort, metabolomic profiling was performed on an additional 1,561 randomly selected subjects from the Framingham Offspring cohort. As expected, the individuals in the extended sample had a lower mean fasting glucose and BMI, compared with the original case-control samples (shown in Table 1). In multivariable Cox regression analyses adjusted for age, gender, fasting glucose, and body mass index, 2-AAA levels remained associated with future diabetes development (adjusted odds ratio, 1.4, per SD increment, p=0.0003). The results were unchanged when models were further adjusted for estimated glomerular filtration rate.

In the whole cohort sample, individuals with 2-AAA values in the highest quartile had an approximately 2-fold risk of developing diabetes, compared with individuals in the lowest quartile (hazard ratio, 2.07, 95% confidence interval, 1.31-3.28). This risk was comparable to that observed in individuals with insulin and HbAlc values in the top quartile, and lower than the risk observed for individuals in the top quartile of BMI or fasting glucose (3- to 4-fold, Table 4).

TABLE 4
Relative risk of diabetes for individuals in the top
quartile of 2- AAA and other metabolic predictors

| | Case-control sample | “Whole cohort” sample |
|---|---|---|
| 2-AAA | 4.56 (1.93-10.75) | 2.07 (1.31-3.28) |
| Insulin | 1.76 (0.97-3.20) | 2.49 (1.56-3.99) |
| Glucose | N/A | 4.23 (2.16-8.40) |
| 2-hour glucose (OGTT) | 2.54 (1.30-5.00) | 3.12 (1.98-4.92) |
| BMI | N/A | 3.34 (1.91-5.84) |
| HbA1c | 1.64 (0.74-3.61) | 2.04 (1.25-3.34) |

Values shown are odds ratios (case-control sample) or hazard ratios (whole cohort sample) from age- and sex-adjusted regression models. 95% confidence intervals are shown in the parentheses.
N/A: not analyzed in the case-control sample because individuals were matched according to fasting glucose and BMI.

Additional adjustment for the presence of prediabetes (defined as hemoglobin Alc 5.7-6.4%, or fasting glucose 100-125 mg/dl) did not alter the results. Separate analyses were also performed in the subgroups of individuals without and with prediabetes to estimate normative values for 2-AAA. A healthy reference sample was selected comprising of individuals from the Framingham Offspring Cohort who met the following criteria: no prior cardiovascular disease, no hypertension, BMI less than 30 kg/m2, no valvular heart disease, and estimated glomerular filtration rate>60. The mean age in the reference sample was 52 years, and 57% were female. Absolute quantitation for 2-AAA was performed using an isotope-labeled reference compound. The median value of 2-AAA in the reference sample was 1.22 M. The full distribution of 2-AAA values in the reference sample is provided in Table 5 below. The findings were similar in individuals without prediabetes (n=781; multivariable-adjusted hazard ratio per SD increment, 1.6, 95% confidence interval 1.04-2.4) and individuals with prediabetes (n=696; 1.3, 1.1-1.6). Normative values for 2-AAA levels in the FHS cohort are detailed in Table 5.

TABLE 5
Distribution of 2-AAA in the reference sample (n = 819)

| Quantile | 2-AAA level (M) |
|---|---|
| 0% (Minimum) | 0.42 |
| 10% | 0.76 |
| 25% Q1 | 0.96 |
| 50% (Median) | 1.22 |
| 75% Q3 | 1.53 |
| 90% | 1.93 |
| 100% (Maximum) | 8.77 |

An important strength of the study discussed herein is the use of two well-characterized longitudinal cohorts with long follow-up periods. All individuals in our study were free of diabetes at the time the blood samples were collected, minimizing potential confounding from medical or lifestyle interventions. Indeed, the data demonstrates that circulating 2-AAA was elevated many years before the onset of diabetes. Furthermore, the relative risk associated with elevated 2-AAA concentrations was not attenuated by adjustment for standard biochemical measures of insulin resistance in the fasting state, or for branched chain and aromatic amino acids, previously validated risk predictors for diabetes.

Follow-up experiments (see examples below) provide evidence that 2-AAA may modulate glucose homeostasis in vivo, while in vitro studies support an effect of 2-AAA on insulin secretion in a pancreatic beta cell line. Taken together, these data highlight a pathway not previously associated with glucose homeostasis, and provide a new metabolic marker to aid in diabetes risk assessment.

In a previous study (8), the present inventors demonstrated that elevated levels of branched chain (isoleucine, leucine, and valine) and aromatic amino acids (phenylalanine and tyrosine) are associated with future diabetes. The relationship between 2-AAA and these metabolites was examined. Concentrations of 2-AAA were poorly correlated with both the branched chain amino acids (r=0.04 to 0.24) and aromatic amino acids (r=0.01 to 0.13). Adjustment for amino acids did not substantially attenuate the association between 2-AAA and future diabetes risk in Framingham or Malmö (data not shown).

2-AAA is generated by lysine degradation, and may also serve as a substrate for enzymes downstream of tryptophan metabolism. Thus, age- and sex-adjusted correlations between 2-AAA and selected metabolites were examined in these pathways. Modest correlations were noted between 2-AAA and lysine (r=0.38, p<0.001), kynurenic acid (r=0.19, p<0.001), and anthranilic acid (r=0.27, p<0.001), though only 2-AAA predicted incident diabetes. (data not shown)

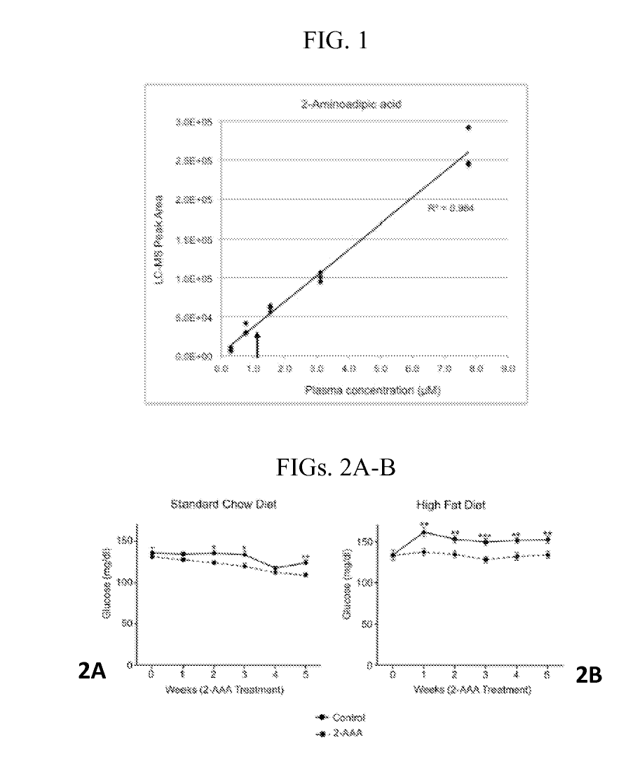

Additional studies were performed with an isotope-labeled reference compound for 2-AAA (d3; C/D/N Isotopes, Inc., Pointe-Claire, Quebec, Canada), the novel biomarker identified. The studies demonstrated that peak areas were greater than two orders of magnitude above the lower limit of quantitation (as defined as a discrete peak 10-fold greater than noise) and fell well within the linear range of the dose-response relationship (FIG. 1). The median level for 2-AAA in the Framingham control population was determined using these data. No quantitative findings in other human populations are available for comparison.

### Example 2

### Effects of a Western-Style Diet on Circulating 2-AAA Levels Mice

The effect of a Western-style diet on circulating 2-AAA levels in mice was examined. C57BL/6 male mice (Jackson Laboratories, Bar Harbor, Me.) were housed in separate cages with free access to food and water. Mice were fed a standard chow diet containing 22.5% protein, 52% carbohydrates, 6% fat, 6% ash and 4% fiber (Prolab Isopro RMH 3000, Brentwood, Mo.) or a high-fat diet containing 20 kcal % protein, 20 kcal % carbohydrate and 60 kcal % fat (DIO formula, D12492, Research Diets, Inc, New Brunswick, N.J.) as indicated. The total energy equivalent was 3.46 kcal/gm for the standard chow diet and 5.24 kcal/gm for the high fat diet.

Animals fed a high-fat diet (HFD) had a 33% increase in baseline glucose concentrations and a 17% increase in insulin levels after 4 weeks. Circulating 2-AAA levels were 51% higher in animals on a HFD compared with those fed the standard chow diet (SCD) (n>11 mice per group, p=0.01). Using an isotopically labeled standard and mass spectrometry, we verified that the 2-AAA content was negligible in both the HFD and SCD (data not shown).

### Example 3

### Role of 2-AAA on Glucose Homeostasis in Mice

Intervention studies in mice were completed to examine whether 2-AAA might play a contributory or compensatory role in glucose homeostasis by performing 2-AAA. For this study, four independent cohorts of 24 C57B/L6 male mice entered the study protocol at 6 weeks of age. Two cohorts received the standard chow diet and two cohorts received a high-fat diet. Half of the mice assigned to each diet received 2-AAA (500 mg/kg/day equivalent to a starting dose of 12.03±0.30 mM) via the drinking water for up to five weeks. Pre-weighed food and water were administered to each cage. Food and water intake was monitored weekly. Mice supplemented with 2-AAA had 33% higher plasma levels of this metabolite by one week of treatment (p=0.018).

Fasting insulin levels in mice were measured by an ELISA kit (Crystal Chem Inc., Downers Grove Ill.). After 5 weeks of 2-AAA treatment, and following a 6-hour fast, each group of mice was administered an intra-peritoneal glucose tolerance test (IPGTT; 1.5 mg/g of body weight; 75 mg/mL of glucose solution) or an insulin tolerance test (ITT; 0.00075 U of insulin/g of body weight, 0.15 U/ml insulin solution, Sigma-Aldrich, St. Louis, Mo.). For the IPGTT, venous blood samples were obtained from the tail vein immediately prior to glucose injection and then serially at 30, 60, and 120 minutes following the injection. For the ITT, venous blood samples were obtained from the tail vein immediately prior to the insulin injection and then serially at 15, 30, 45 and 60 minutes following the injection.

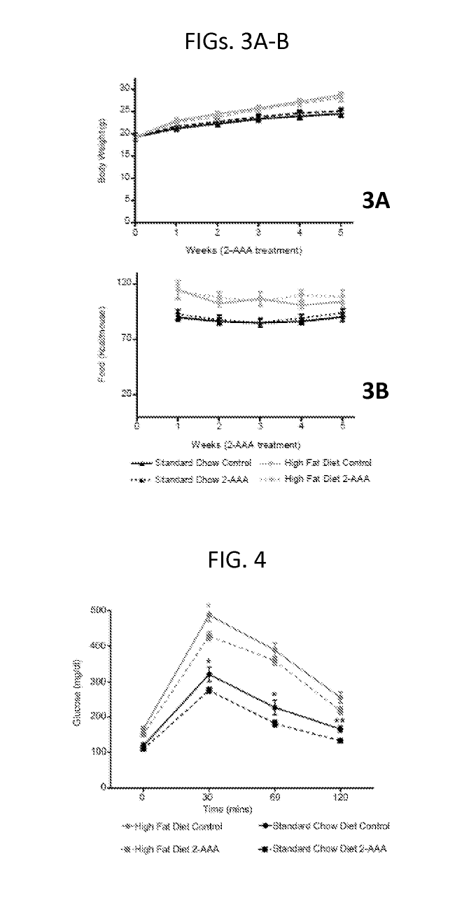

Fasting glucose levels in the 2-AAA treated mice were consistently lower baseline on both diets (p<0.001 by 2-way ANOVA analysis after 5 weeks; FIGS. 2A-B). Fasting plasma glucose levels were measured weekly in mice fed either a standard chow (left) or high-fat diet (right) beginning at 6 weeks of age, with simultaneous 2-AAA treatment via drinking water (500/mg/kg/day) or water alone for the subsequent 5 weeks. (n=24 mice per condition) (*p<0.05; **p<0.01; ***p<0.001). For mice on the SCD, fasting glucose levels were 109.5±3.8 mg/dL for the 2-aminoadipic treated animals as compared to 124.5±4.9 mg/dL, for the untreated controls after 5 weeks (p<0.01, FIG. 2A). For mice challenged with a HFD, fasting glucose levels were higher and the differences due to 2-AAA treatment were accentuated (134.5±5.9 vs. 153.0±6.0 vs. mg/dL at 5 weeks; p<0.01; FIG. 2B). There were no significant differences in food intake or weight between treated and control mice (FIG. 3).

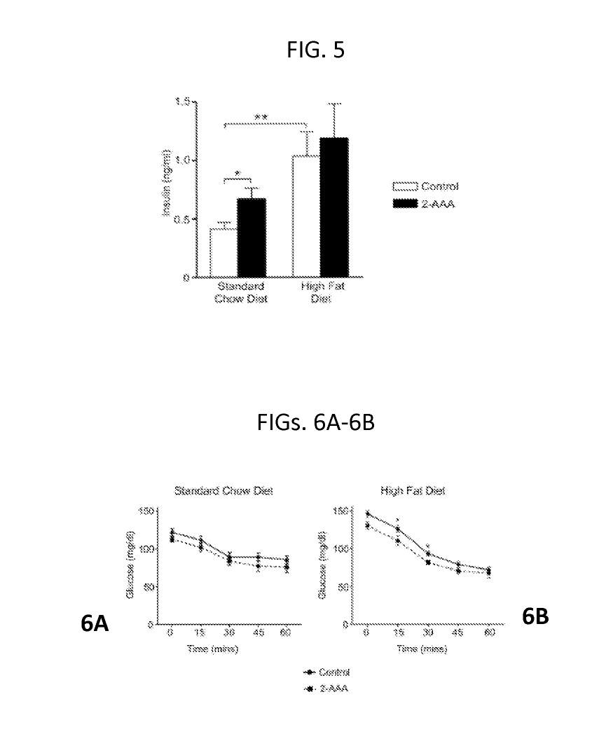

Additional studies were performed using acute physiologic challenges, including acute glucose and insulin administration. As expected, mice fed a HFD had more pronounced glucose excursions following the glucose challenge (FIG. 4). In mice fed both the SCD and HFD for 5 weeks or greater, peak glucose concentrations following the glucose challenge were lower in the 2-AAA treated mice. Increases in fasting insulin levels were observed in the HFD animals as compared to the SCD controls (1.040±0.203 vs. 0.411±0.061 ng/mL, respectively; p=0.013), which was further augmented by the administration of 2-AAA (FIG. 5). Following acute insulin challenge, 2-AAA had no effect on the rate of decline in glucose levels (FIGS. 6A and 6B) indicating no difference in peripheral insulin sensitivity. Taken together, these findings highlight a role for 2-AAA in modulating glucose levels in vivo. 2-AAA treatment appears to augment circulating insulin concentrations, without altering peripheral insulin resistance.

Results for biochemical measures of insulin resistance and -cell function are shown in Table 6. Fasting concentrations of 2-AAA were moderately correlated with fasting insulin (age- and sex-adjusted partial correlation, r=0.25; p<0.001), HOMA-IR (r=0.24; p<0.001), HOMA-B (r=0.25, p<0.001) and 2-hour glucose during oral glucose tolerance testing (r=0.14; p=0.006). Baseline concentrations of 2-AAA and hemoglobin Alc were not significantly correlated (r=0.05; p=0.37), consistent with the non-diabetic status of all individuals at baseline. The association of 2-AAA level and incident diabetes was unchanged even after adjusting for these measures of insulin resistance and beta cell function (Table 4). There were also no significant associations between 2-AAA and dietary intake of fat, protein, carbohydrates, or lysine (data not shown).

TABLE 6
Biochemical measures of glycemia in study samples

| | Framingham Heart Study | | Malmö Diet and Cancer Study | |
|---|---|---|---|---|
| | | Matched | | Matched |
| | Cases | Controls | Cases | Controls |
| | (n = 188) | (n = 188) | (n = 162) | (n = 162) |
| Fasting | 105 (14) | 106 (12) | 97 (13) | 97 (11) |
| glucose, | | | | |
| (mg/dl) | | | | |
| Hemoglobin | 5.5 (0.7) | 5.4 (0.8) | — | — |
| A1c, (%) | | | | |
| Fasting insulin, | 11.7 (11.4) | 9.9 (9.6) | 9.0 (6.0) | 9.0 (6.0) |
| (uIU/ml) | | | | |
| HOMA-IR | 3.0 (2.8) | 2.5 (2.6) | 2.2 (1.4) | 2.1 (1.7) |
| 2-hour OGTT | 123 (44) | 115 (39) | — | — |
| glucose, | | | | |
| (mg/dl) | | | | |
| Prediabetes, % | — | 83% | — | — |

### Example 4

### Tissue Distribution of 2-AAA

To better understand the source of 2-AAA and the organ in which it might be playing a functional role, LC-MS/MS was used to measure 2-AAA levels in metabolically active tissue (muscle, liver, fat, and pancreas). Tissues were harvested from mice at baseline and following the chronic administration of 2-AAA, on either a SCD or a HFD metabolite profiling analysis. For homogenization of liver and pancreas, 25 mg of tissue sample were mixed with 250 μl of a 50:50 MeOH:H20 solution. For the skeletal muscle, 25 mg of tissue were mixed with 250 μL of HPLC water (J. T. Baker, Center Valley Pa.). All tissue samples were then homogenized for 4 minutes at 25 Hz in a TissueLyser II (Qiagen, Hilden, Germany). 200 μL of the resulting homogenates were extracted following a modified Bligh-Dyer method (17), and the resulting aqueous phase was dried down and reconstituted in methanol containing labeled isotope standards (L [symbol] Phenylalanine-d8 and L-Valine-d8) as performed with the plasma samples.

For the perigonadal adipose tissue, metabolites were first extracted by mixing harvested tissues with 6 μL per 1 mg of adipose tissue of a MeOH:Chloroform solution (2:1 v/v). The extracted adipose tissues were then homogenized for 4 minutes at 25 Hz in a TissueLyser II. The resulting homogenates were mixed with chloroform and water (2 μL per 1 mg of adipose tissue for each solvent) and centrifuged at 14,000 rpm for 20 minutes at 4° C. 2 μL per mg of tissue of the upper aqueous layer were dried down and reconstituted in a methanol solution containing labeled standards (L-Phenylalanine-d8 and L-Valine-d8), as previously described (24). A calibration curve using 2-AAA d3 (C/D/N Isotopes Inc, Quebec Canada) was generated for absolute quantitation of 2-AAA in plasma and tissue samples. LC-MS/MS analyses were then performed using the same methodology as described above for human plasma. An isotopically labeled standard was used to facilitate absolute quantitation of the metabolite of interest in the setting of the different biological matrices.

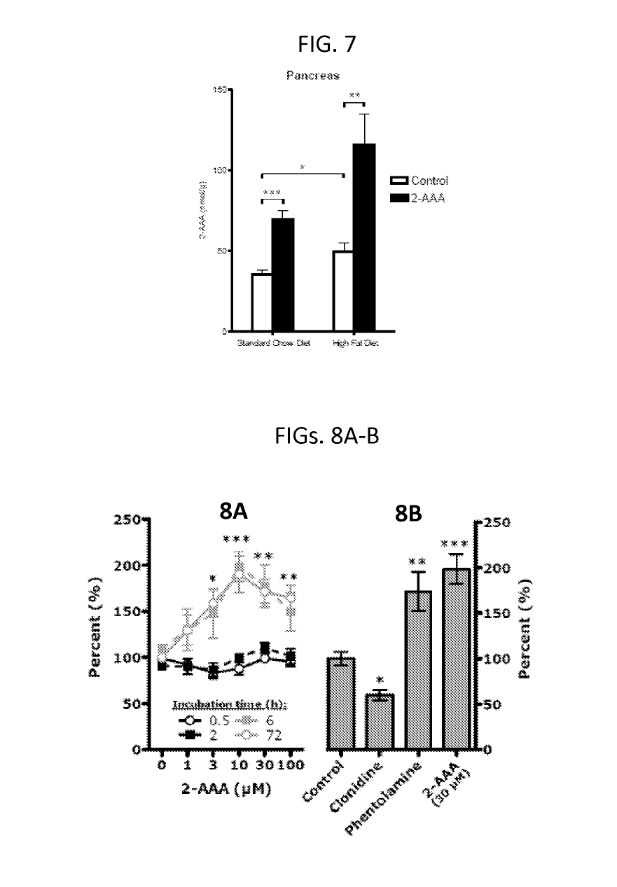

These studies demonstrated that 2-AAA was most abundant in the pancreas, though it was also present in all of the tissues tested in varying amounts. Furthermore, in the pancreas alone higher 2-AAA levels were documented following the administration of the HFD as compared to SCD (35.54±2.54 vs. 49.31±5.75 nmol/g tissue, p<0.05), as well as a striking increase in 2-AAA levels following 2-AAA administration (SCD control vs. SCD treated: 35.54±2.54 vs. 69.4±5.66 nmol/g tissue, p<0.001; HFD control vs. HFD treated: 49.31±5.75 vs. 115.88±18.57 nmol/g tissue, p<0.002; FIG. 7)

### Example 5

### 2-AAA Associated Insulin Production

To investigate the connection between 2-AAA and the pancreas, insulin production by a pancreatic beta cell line that was acutely and chronically exposed to 2-AAA. Beta TC6 (BTC6) cells, an established model to examine insulin secretion (mouse insulinoma beta-TC-6 (ATCC® CRL-11506™) from ATCC (Manassas, Va.)) (27-29), were used at passage number 4-7, grown in DMEM (ATCC 2002-30), 15% FBS, with penicillin/streptomycin (100 IU/ml/100 μg/ml). Cells were plated on 24 well collagen plates at 40,000 cells per well, incubated with 2-AAA at varying concentrations ranging from 0 to 100 μM for 0 to 72 hours to assess whether this compound increases insulin secretion in a time and/or dose dependent fashion. On the day of experimentation, the cells were washed with PBS and the media was changed to DMEM without FBS or glucose to which 0.1% BSA was added. After 1 hour of incubation, this media was changed to serum free media containing 2.5 mM of glucose. Insulin production was measured in the supernatant after 1 additional hour of incubation. To assess the time response relationship, 2-AAA was added to the cells after plating on collagen and incubated for 0.5, 2, 6, and 72 hours.

As demonstrated in FIG. 8A, 2-AAA induced insulin secretion from BTC6 cells in a dose and time dependent fashion. The extent of 2-AAA (30 μM) stimulated insulin secretion was then compared to the effects of clonidine (100 μM) and phentolamine (100 μM), which inhibit and stimulate insulin secretion in islet cells, respectively (FIG. 8B). Augmented insulin secretion was evident with at least 6 hours of incubation with 2-AAA. Further, the concentrations used to elicit secretion were in the physiologic range. By way of comparison, clonidine (a known inhibitor of insulin secretion) decreased insulin levels to 60±3% of control, phentolamine (a known potent stimulator) increased insulin secretion to 172±8% of control, which was comparable to the peak secretion triggered by 2-AAA (FIG. 8B).

All animal experiments were performed in accordance with protocols approved by the Subcommittee on Research Animal Care at the Massachusetts General Hospital.

### Example 6

### 2-AAA Induces Insulin Secretion from Mammalian Islets

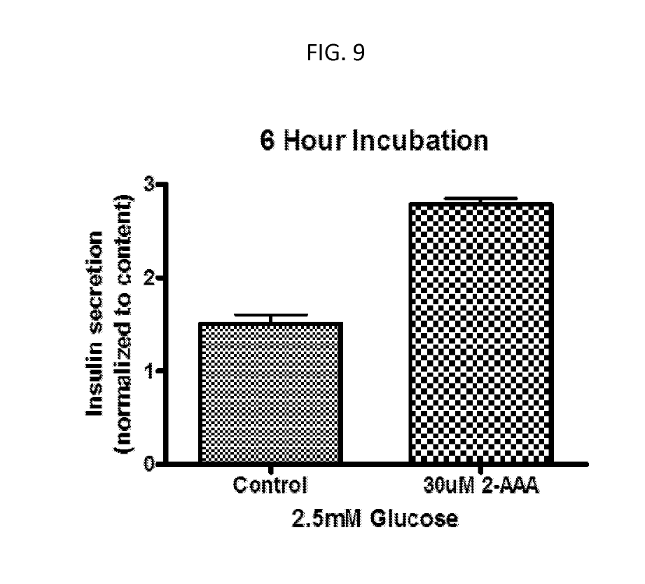

Islets from male C57BL/6J mice were isolated by collagenase digestion of the pancreas, purified by Ficoll density gradient and then handpicked. Mouse islets were cultured for 24 hours as previously described (26). For insulin secretion experiments, 15 islets were placed in each microcentrifuge tube and incubated in islet secretion buffer containing (in mmol/1) 120 NaCl, 5 KCl, 1 CaCl2, 1.2 MgCl2, 24 NaHCO3, 10 HEPES, and 2.5 glucose, bubbled with 95% O2/5% CO2 and supplemented with 0.5% (wt/vol) BSA. Experiments were performed by incubating islets in 1 ml of secretion buffer in the presence or absence of 30 μM 2-AAA for 6 hours at 37° C., 5% CO2. Insulin was assayed using the Meso Scale Discovery multi array assay system for mouse/rat total insulin (Gaithersburg, Md., USA). Secretion was normalized to islet content. FIG. 9 demonstrates that islets incubated in the presence of 30 μM 2-AAA release insulin.

### REFERENCES

1. Pan, X. R., Li, G. W., Hu, Y. H., Wang, J. X., Yang, W. Y., An, Z. X., Hu, Z. X., Lin, J., Xiao, J. Z., Cao, H. B., et al. 1997. Effects of diet and exercise in preventing NIDDM in people with impaired glucose tolerance. The Da Qing IGT and Diabetes Study. Diabetes Care 20:537-544.
2. Tuomilehto, J., Lindstrom, J., Eriksson, J. G., Valle, T. T., Hamalainen, H., Ilanne-Parikka, P., Keinanen-Kiukaanniemi, S., Laakso, M., Louheranta, A., Rastas, M., et al. 2001. Prevention of type 2 diabetes mellitus by changes in lifestyle among subjects with impaired glucose tolerance. N. Engl. J. Med. 344:1343-1350.
3. The Diabetes Prevention Program Research Group. 2002. Reduction in the incidence of type 2 diabetes with lifestyle intervention or metformin. N Engl J Med 346:393-403.
4. Gerstein, H. C., Yusuf, S., Bosch, J., Pogue, J., Sheridan, P., Dinccag, N., Hanefeld, M., Hoogwerf, B., Laakso, M., Mohan, V., et al. 2006. Effect of rosiglitazone on the frequency of diabetes in patients with impaired glucose tolerance or impaired fasting glucose: a randomised controlled trial. Lancet 368:1096-1105.
5. Gieger, C., Geistlinger, L., Altmaier, E., Hrabe de Angelis, M., Kronenberg, F., Meitinger, T., Mewes, H. W., Wichmann, H. E., Weinberger, K. M., Adamski, J., et al. 2008. Genetics meets metabolomics: a genome-wide association study of metabolite profiles in human serum. PLoS Genet 4:e1000282.
6. Suhre, K., Meisinger, C., Doring, A., Altmaier, E., Belcredi, P., Gieger, C., Chang, D., Milburn, M. V., Gall, W. E., Weinberger, K. M., et al. Metabolic footprint of diabetes: a multiplatform metabolomics study in an epidemiological setting. PLoS One 5:e13953.
7. Suhre, K., Shin, S. Y., Petersen, A. K., Mohney, R. P., Meredith, D., Wagele, B., Altmaier, E., Deloukas, P., Erdmann, J., Grundberg, E., et al. Human metabolic individuality in biomedical and pharmaceutical research. Nature 477:54-60.
8. Wang, T. J., Larson, M. G., Vasan, R. S., Cheng, S., Rhee, E. P., McCabe, E., Lewis, G. D., Fox, C. S., Jacques, P. F., Fernandez, C., et al. 2011. Metabolite profiles and the risk of developing diabetes. Nat Med 17:448-453.
9. Rhee, E. P., Cheng, S., Larson, M. G., Walford, G. A., Lewis, G. D., McCabe, E., Yang, E., Farrell, L., Fox, C. S., O'Donnell, C. J., et al. Lipid profiling identifies a triacylglycerol signature of insulin resistance and improves diabetes prediction in humans. J Clin Invest 121:1402-1411.
10. Requena, J. R., Chao, C. C., Levine, R. L., and Stadtman, E. R. 2001. Glutamic and aminoadipic semialdehydes are the main carbonyl products of metal-catalyzed oxidation of proteins. Proc Natl Acad Sci USA 98:69-74.
11. Fan, X., Zhang, J., Theves, M., Strauch, C., Nemet, I., Liu, X., Qian, J., Giblin, F. J., and Monnier, V. M. 2009. Mechanism of lysine oxidation in human lens crystallins during aging and in diabetes. J Biol Chem 284:34618-34627.
12. Sell, D. R., Strauch, C. M., Shen, W., and Monnier, V. M. 2007. 2-aminoadipic acid is a marker of protein carbonyl oxidation in the aging human skin: effects of diabetes, renal failure and sepsis. Biochem J 404:269-277.
13. Tsutsui, H., Maeda, T., Toyo'oka, T., Min, J. Z., Inagaki, S., Higashi, T., and Kagawa, Y. 2010. Practical analytical approach for the identification of biomarker candidates in prediabetic state based upon metabonomic study by ultraperformance liquid chromatography coupled to electrospray ionization time-of-flight mass spectrometry. J Proteome Res 9:3912-3922.
14. Wijekoon, E. P., Skinner, C., Brosnan, M. E., and Brosnan, J. T. 2004. Amino acid metabolism in the Zucker diabetic fatty rat: effects of insulin resistance and of type 2 diabetes. Can J Physiol Pharmacol 82:506-514.
15. Weyer, C., Bogardus, C., Mott, D. M., and Pratley, R. E. 1999. The natural history of insulin secretory dysfunction and insulin resistance in the pathogenesis of type 2 diabetes mellitus. J Clin Invest 104:787-794.
16. Felig, P., Marliss, E., and Cahill, G. F., Jr. 1969. Plasma amino acid levels and insulin secretion in obesity. N Engl J Med 281:811-816.
17. Wahren, J., Felig, P., Cerasi, E., and Luft, R. 1972. Splanchnic and peripheral glucose and amino acid metabolism in diabetes mellitus. J Clin Invest 51:1870-1878.
18. Wahren, J., Felig, P., and Hagenfeldt, L. 1976. Effect of protein ingestion on splanchnic and leg metabolism in normal man and in patients with diabetes mellitus. J Clin Invest 57:987-999.
19. Liu, Z., Jeppesen, P. B., Gregersen, S., Chen, X., and Hermansen, K. 2008. Dose- and Glucose-Dependent Effects of Amino Acids on Insulin Secretion from Isolated Mouse Islets and Clonal INS-1E Beta-Cells. Rev Diabet Stud 5:232-244.
20. Um, S. H., D'Alessio, D., and Thomas, G. 2006. Nutrient overload, insulin resistance, and ribosomal protein S6 kinase 1, S6K1. Cell Metab 3:393-402.
21. Newgard, C. B., An, J., Bain, J. R., Muehlbauer, M. J., Stevens, R. D., Lien, L. F., Haqq, A. M., Shah, S. H., Arlotto, M., Slentz, C. A., et al. 2009. A branched-chain amino acid-related metabolic signature that differentiates obese and lean humans and contributes to insulin resistance. Cell Metab 9:311-326.
22. Kannel, W. B., Feinleib, M., McNamara, P. M., Garrison, R. J., and Castelli, W. P. 1979. An investigation of coronary heart disease in families. The Framingham offspring study. Am J Epidemiol 110:281-290.
23. Persson, M., Hedblad, B., Nelson, J. J., and Berglund, G. 2007. Elevated Lp-PLA2 levels add prognostic information to the metabolic syndrome on incidence of cardiovascular events among middle-aged nondiabetic subjects. Arterioscler Thromb Vasc Biol 27:1411-1416.
24. Roberts, L. D., Souza, A. L., Gerszten, R. E., and Clish, C. B. Targeted metabolomics. Curr Protoc Mol Biol Chapter 30:Unit 30 32 31-24.
25. Matthews, D. R., Hosker, J. P., Rudenski, A. S., Naylor, B. A., Treacher, D. F., and Turner, R. C. 1985. Homeostasis model assessment: insulin resistance and beta-cell function from fasting plasma glucose and insulin concentrations in man Diabetologia 28:412-419.
26. Hamming K S, Soliman D, Webster N J, Searle G J, Matemisz L C, Liknes D A, Dai X Q, Pulinilkunnil T, Riedel M J, Dyck J R, Macdonald P E, Light P E: Inhibition of beta-cell sodium-calcium exchange enhances glucose-dependent elevations in cytoplasmic calcium and insulin secretion. Diabetes 59:1686-1693, 2010.
27. Poitout V, Stout L E, Armstrong M B, Walseth T F, Sorenson R L, and Robertson R P., Morphological and functional characterization of beta TC-6 cells—an insulin-secreting cell line derived from transgenic mice. Diabetes. 1995 March; 44(3):306-13.
28. Laurance M E, et al. Glucose responsive insulin secreting beta-cell lines and method for producing same. U.S. Pat. No. 5,773,255 dated Jun. 30, 1998.
29. Poitout V, et al. Insulin-secreting cell lines: classification, characteristics and potential applications. Diabetes Metab. 22: 7-14, 1996.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1.-9. (canceled)

10. A method for determining risk of developing diabetes in a subject, the method comprising:
determining a level of 2-aminoadipic acid (2-AAA) in a test sample from the subject;
comparing the level of 2-AAA in the test sample to a reference level; and
determining the subject has an increased risk of developing diabetes when the test sample has an increased level of 2-AAA as compared to the reference level.

11. The method of claim 10, wherein the test sample comprises serum or plasma from the subject.

12. The method of claim 10, wherein the subject has normal glucose tolerance.

13. The method of claim 10, further comprising selecting a treatment based on the level of 2-aminoadipic acid in the test sample.

14. The method of claim 13, further comprising administering the selected treatment to the subject.

15. The method of claim 13, wherein the treatment comprises administering to the subject an effective amount of at one or more additional anti-diabetes compound selected from the group consisting of acarbose, miglitol, metformin, phenformin, buformin, repaglinide, nateglinide, tolbutamide, chlorpropamide, tolazamide, acetohexamide, glyburide, glipizide, glimepiride, gliclazide, troglitazone, rosiglitazone, pioglitazone, peptide analogs, glucagon-like peptide I (GLP1) and analogs thereof, GLP agonists, vildagliptin sitagliptin; dichloroacetic acid; amylin, carnitine palmitoyltransferase inhibitors, B3 adrenoceptor agonists, and insulin.

16. The method of claim 13, wherein the treatment comprises administering to the subject an effective amount of 2-aminoadipic acid for increasing the level of insulin secretion.

17. The method of claim 10, wherein the subject has at least one risk factor for diabetes.

18. The method of claim 10, wherein the level of 2-aminoadipic acid is determined using a mass spectrometer.

19. The method of claim 10, the method further comprising determining the level of 2-AAA in a control sample from a control subject not having, or at risk of developing diabetes;
comparing the level of 2-AAA in the test sample to the level of 2-AAA in the control sample; and
determining the subject has an increased risk of developing diabetes when the test sample has an increased level of 2-AAA as compared to the level of 2-AAA in the control sample.

20. A kit for use in a method of determining risk of diabetes in a subject of claim 10, the kit comprising one or more control samples comprising predetermined levels of 2-aminoadipic acid.

21. A method for the treatment of a glucose-related metabolic disorder, comprising administering a therapeutically effective amount of 2-aminoadipic acid (2-AAA).

22. The method according to claim 21, wherein 2-AAA is administered in a pharmaceutical composition.

23. The method according to claim 22, wherein the pharmaceutical composition is administered in the form of tablets, granules, capsules, suspensions, solutions or injections.

24. The method according to claim 21, wherein the glucose-related metabolic disorder is diabetes or Metabolic Syndrome.

25. The method according to claim 24, wherein said diabetes is type 1 diabetes, type 2 diabetes, or gestational diabetes.

26. The method according to claim 21, the method further comprising administering one or more additional anti-diabetes compounds selected from the group consisting of acarbose, miglitol, metformin, phenformin, buformin, repaglinide, nateglinide, tolbutamide, chlorpropamide, tolazamide, acetohexamide, glyburide, glipizide, glimepiride, gliclazide, troglitazone, rosiglitazone, pioglitazone, peptide analogs, glucagon-like peptide I (GLP1) and analogs thereof, GLP agonists, vildagliptin sitagliptin; dichloroacetic acid; amylin, carnitine palmitoyltransferase inhibitors, B3 adrenoceptor agonists, and insulin.
